(19) United States
(12) Patent Application Publication
(10) Pub. No.: US 2010/0016991 A1
(43) Pub. Date: Jan. 21, 2010

(54) Prosthetic product having composite material wall, and method for producing the prosthetic product

(76) Inventor: Kennet Hellberg, Vallentuna (SE)

Correspondence Address: KEVIN FARRELL;PIERCE ATWOOD, ONE NEW HAMPSHIRE AVENUE, PORTSMOUTH, NH, 03801, US

(21) Appl. No.: 12/311,085

(22) PCT Filed: Sep. 5, 2007
(86) PCT No.: PCT/SE2007/050623

§ 371 Date: Mar. 18, 2009

(30) Foreign Application Priority Data
Sep. 19, 2006 (SE) 0601943-4

(51) Int. Cl. A61F 2/60; B29C 70/34; B29C 70/44; B29C 70/06; A61F 2/54; A61F 2/00

(52) U.S. Cl. 623/33; 264/258; 264/101; 623/57

(57) ABSTRACT

The invention relates to a prosthetic product having a composite material wall moulded into the shape of a part of a body, characterized in that the wall of the prosthetic product is composed of, on one hand, an reinforcing textile including continuous length fibres, and on the other hand, a textile matrix including thermoplastic polymer fibres, preferably of continuous length. Analogously herewith, the invention also relates to a process for the manufacture of a prosthetic product comprising a composite material wall moulded into the shape of a part of a body, comprising the following manufacturing steps: providing a mould onto which the composite material wall is to be formed; applying an reinforcing textile including continuous length fibres onto the mould; applying a matrix textile including thermoplastic polymer fibres preferably of continuous length onto the mould, at least externally of the reinforcing textile, and melting the polymer matrix fibres for embedding of the reinforcing textile fibres in a thermoplastic polymer matrix, followed by cooling and consolidation of the composite material wall.

### TECHNICAL FIELD OF THE INVENTION

The invention relates to a prosthetic product having a wall made of composite material being formed on a casting from a part of the body. The invention also relates to method for producing a composite material wall included in such prosthetic product, as well as the use of a hose-shaped fibre textile for producing the prosthetic product.

### BACKGROUND AND PRIOR ART

As used herein, prosthetic product mainly refers to prosthesis or parts of prosthesis which are shaped as substitutes for a lost part of the body, such as an arm or a leg. That will not exclude the advantageous implementation of the invention in connection with orthopaedic aids such as orthoses that are shaped for supporting a weak, injured or disfigured body part, or in connection with inserts such as shoe inserts which are shaped for correction of an incorrect position of the foot, for example.

A common feature for the prosthetic products is that the same are shaped on a casting of the subject part of the body. The casting is a three step process wherein a first casting results in a negative cast of the body part, where after a positive copy is moulded from the negative cast. Finally, the prosthetic product is cast on the positive copy, to which purpose two basically different polymer materials and techniques are typically used: on one hand the curing plastics which set into a definite and irrevocable shape, and on the other hand the thermoplastics which after heating and cooling permit a correction of the shape of the prosthetic product by repeated heating and cooling.

In the first mentioned moulding technique a reinforcing textile is usually applied onto the positive copy, whereupon the textile is impregnated with a liquid curing plastic which forms, upon curing and possibly at a raised temperature, a composite shell about the positive copy.

The invention is to be referred to the last mentioned moulding technique, resulting in a prosthetic product which can be afterwards customized.

The ability of a thermoplastic material to repeatedly transform into a shaping phase is essential for the wearer of a prosthetic device in the form of a sleeve, or an orthosis or an insert. A body part, which carries or is supported by a prosthetic product, changes over time, on one hand at a longer perspective with respect to volume/size, and on the other hand on a shorter perspective with respect to swelling or muscle movements. In many cases this requires an afterwards adjustment of the shape of a prosthetic product which initially is produced for a body at rest.

The use of thermoplastics for production of prosthetic products has hitherto and typically included heating of sheathed thermoplastic polymers, wherein the sheaths have in advance been mounted in rigid frames. The sheath, which is softened through the heating, is drawn onto the positive copy and left to cure into the shape of the mould, in a consolidation process. Eventually, consolidation proceeds at elevated temperature and under the application of a pressure from outside the prosthetic product.

Besides the already mentioned moulding techniques there is also practised lamination of a composite wall onto the positive mould, wherein fibre mats that are impregnated with partly cured thermosetting plastics are trimmed and heated from a cold store temperature so as to be deformable enough to permit manual shaping onto the positive mould. A laminate formed by these so called prepreg materials is usually left for consolidation under pressure and at elevated temperatures.

In all cases, heating of the polymer matrix can be performed in an oven as air is simultaneously evacuated from the composite material, usually by means of a vacuum bag which is connected to a pump and which encloses the positive mould and the composite material.

All the known methods involve a manual work which is not only laborious and executed against the clock, but which also requires skill and experience, and which further is messy and performed in a work environment which is loaded with substances that carry nasty fumes and irritates the skin, especially in connection with thermosetting plastics. Another problem connected with known methods and materials comprised in the technical field of the invention is the difficulty, upon moulding the material onto the positive mould, to determine the fibre direction of reinforcing textiles included in the composite material. The direction of reinforcement fibres may be critical for the strength in the finished product, or for the possibility to control flexibility and rigidity in the product. Furthermore, the shaping of thermoplastic sheaths or prepreg material into prosthetic products results in considerable amounts of waste material.

### SUMMARY OF THE INVENTION

The present invention provides a solution to these problems. One object for the invention is to provide a prosthetic product having a composite material wall which can be produced in a simplified manufacturing process.

Another object for the invention is to provide a prosthetic product which can be produced in an improved and healthy work environment.

Yet another object for the invention is to provide a prosthetic product having a fibre reinforced composite material wall offering both high strength and low weight.

Still another object for the invention is to provide a prosthetic product having a fibre reinforced composite material wall offering both high strength and low weight and which is repeatedly formable in adaptation to the wearer.

A further object for the invention is to provide a production process which results in a prosthetic product comprising the qualities mentioned above.

A still further object for the invention is to provide a production process which results in reduced consumption and reduced waste of material.

It is a still further object for the invention to provide a production process which permits complete control of the fibre direction of reinforcement fibres in the finished prosthetic product.

The above object or objects are achieved in a prosthetic product composed in accordance with the characterizing part of claim 1, and in a production process for manufacture of the prosthetic product according to the invention. Advantageous embodiments of the product and process are defined in the subordinated claims.

Briefly, by the present invention there is provided a prosthetic product having a composite material wall moulded into the shape of a part of a body, characterized in that the wall of the prosthetic product is composed of, on one hand, an reinforcing textile including continuous length fibres, and on the other hand, a textile matrix including thermoplastic polymer fibres, preferably of continuous length, which are solidified together into a composite on a positive copy of the body part and under the supply of heat and pressure. The reinforcing textile and the thermoplastic polymer matrix textile may both include fibres which are interlaced, or woven, or knitted into a textile which is elastically extendable in at least one direction of the textile.

The composite material wall of the prosthetic product is preferably composed of between 30 and 80 percents by volume of reinforcing fibres, and between 70 and 20 percents by volume of thermoplastic polymer matrix fibres. Reinforcing fibres may preferably be included in the amount of between 40 and 80 percents by volume, and the thermoplastic polymer fibres in the amount of between 60 and 20 percents by volume.

In one embodiment, the reinforcing fibres and the thermoplastic fibres are woven or knitted together. An advantageous embodiment foresees that the fibres are woven or knitted into a circular composite textile.

In another embodiment the reinforcing textile and polymer matrix textile both include continuous fibres of thermoplastic polymer. In this case, the thermoplastic fibres of the reinforcing textile have a higher melting temperature than that of the polymer matrix textile.

In one embodiment the fibres of the reinforcing textile include mainly at least one fibre from the group of fibres including glass fibres, carbon fibres or aramnid fibres. In said embodiment, the fibres of the polymer matrix textile include mainly at least one fibre from the group of fibres including ethylene plastics, polyester plastics, vinyl plastics or other thermoforming plastics.

The composite material wall of the prosthetic product includes at least one reinforcing textile that is embedded between thermoplastic polymer matrix textiles.

To meet requests for strength and wall thickness, the composite material wall may be composed from multiple layers, whereupon every other layer may include a reinforcing textile and the layer between includes a polymer matrix textile. It is also conceivable to arrange the included layers differently with respect to shape of fibres, fibre dimension, fibre density, or any other controllable parameter which is of importance for the properties of the finished composite material wall.

At least one of the reinforcing textile and the polymer matrix textile can be knitted to a shape. In such embodiment, one or both of the reinforcing textile and the polymer matrix textile may have, in a local area thereof, a number of meshes that differ from the number of meshes in other parts of the textile. If appropriate, at least one of the reinforcing textile and the polymer matrix textile may have a growing number of meshes in at least one direction of the textile.

In an embodiment which is knitted into shape, one or both of the reinforcing textile and the polymer matrix textile can have, in a local area, a mash size that differs from the mesh size in other parts of the textile.

In an embodiment which is knitted into shape, one or both of the reinforcing textile and the polymer matrix textile can have, in a local area, a higher weight per area then in other parts of the textile. In such embodiment, at least one of the reinforcing textile and the polymer matrix textile may include individual fibres of wider diameter then the other fibres in the textile, or individual strings of fibres comprising larger number of individual fibres than in other parts of the same.

An embodiment that is knitted into shape may also include that one or both of the reinforcing textile and the polymer matrix textile in a local area has a higher fibre density than in other parts of the textile. In such embodiment, at least one of the reinforcing textile and the polymer matrix textile can have a higher fibre density in an axially limited portion about the textile. Alternatively, or in combination therewith, one or both of the reinforcing textile and the polymer matrix textile can have a higher fibre density in a circumferentially limited axial portion of the textile.

The prosthetic product is usually structured to resist loads in form of pressure, and/or tension, and/or bending, and/or shear, whereto the reinforcing textile advantageously has a first fibre direction extending mainly in the line of force of the main load which is applied to the prosthetic product in use, and a second fibre direction mainly extending transversally to the line of force of the main load which is applied to the prosthetic product in use. Alternatively, the reinforcing textile may have first and second fibre directions extending angularly relative to the line of force of the load which is applied to the prosthetic product in use.

At least one of the textiles, preferably both, may advantageously be woven, knitted or interlaced circular, into the shape of a hose. In such embodiment, at least one of the reinforcing textile and the polymer matrix textile may disclose more elasticity transversely to the hose than in the axial direction thereof. At least on of the reinforcing textile and the polymer matrix textile, preferably both of them may be of a non-specified length.

An embodiment which is knitted into a shape includes that one or both of the reinforcing textile and the polymer matrix textile is woven/knit into the shape of a curved stocking, having one or two open ends.

According to invention a process is also provided for the manufacture of a prosthetic product comprising a composite material wall moulded into the shape of a part of a body.

The process comprises the following manufacturing steps:
a providing a mould onto which the composite material wall is to be formed;
applying an reinforcing textile including continuous length fibres onto the mould;
applying a matrix textile including thermoplastic polymer fibres preferably of continuous length onto the mould, at least externally of the reinforcing textile, and
melting the polymer matrix fibres for embedding of the reinforcing textile fibres in a thermoplastic polymer matrix, followed by cooling and consolidation of the composite material wall.

Advantageously, thermoplastic polymer matrices are applied onto the mould both externally as well as internally of the reinforcing textile. The composite material wall of the prosthetic product is also advantageously formed by applying alternating layers of reinforcing textile and thermoplastic polymer matrix textile onto the mould in the construction of a multi layered prosthetic product wall, which through melting and cooling of the polymer matrix textile is consolidated into a homogenous composite material wall having the shape of the mould.

Consolidation of the composite material wall includes that the thermoplastic polymer matrix textile is melted while pressure is applied there about to act inwardly, towards the mould. The pressure applies typically includes a negative pressure which is generated inside an elastic sleeve that is arranged to enclose the mould and the armour and polymer matrix textiles, respectively, applied onto the mould. A sleeve that is suitable for this purpose may be formed from silicon.

The process advantageously includes that an elastic sleeve of air permeable material or an air permeable textile is applied onto the mould, internally of the textiles of reinforcing fibres and thermoplastic polymer fibres, respectively, the process also advantageously includes that an elastic spacer sleeve is applied onto the mould, internally of the textiles of reinforcing fibres and thermoplastic polymer fibres, respectively, and externally and/or internally of the air permeable sleeve. Also the spacer sleeve may be formed from silicon.

An advantageous embodiment of the process is characterized in that the textiles applied onto the mould are shaped as hoses.

### SHORT DESCRIPTION OF THE DRAWINGS

The invention will be more fully explained below with reference made to the accompanying schematic drawings, wherein

FIGS. 1a and 1b illustrates, in simplified manner, the manufacture of a mould in the form of a positive copy of a part of a body;

FIG. 2 illustrates schematically the building up of a composite material wall on the mould;

FIG. 3 illustrates schematically a set up for the consolidation of a composite material wall of a prosthetic product according to the invention, in this case a prosthetic sleeve;

FIG. 4 illustrates schematically the manufacture of another prosthetic product, in this case an orthosis, and

FIG. 5 illustrates schematically the manufacture of yet another prosthetic product, in this case an insert.

### DETAILED DESCRIPTION OF PREFERRED EMBODIMENTS

The expression “prosthetic product” shall here be understood to encompass a prosthesis or a part of a prosthesis, intended as a substitute for a missing part of a body, in such application, the prosthetic product maybe shaped as a rigid sleeve adapted to an amputated limb, and to which an artificial leg or arm can be connected. The prosthetic product may also form a rigid sleeve to which an artificial hand or foot can be connected. Albeit this is the main field of application for a prosthetic product, the advantages provided thereby can at least partially be achieved also in other types of products within this field of technology, such as in supporting inserts or orthoses.

The expression “textile” shall here be understood to encompass a flexible material comprising a network of natural or artificial fibres, which can be interlaced, woven or knitted, or compressed to provide an interlocked network. A textile suitable for the purpose of the invention is a textile wherein fibres are interlaced, or woven, or knitted to form an elastically extensible textile, with a marked direction of flexibility transversally to, along with, or at an angle to the length of the textile.

The expression “composite” shall here be understood to encompass a composite or synthetic material including at least two materials of differing properties. In this connection, more specifically, the expression refers to a material including reinforcing fibres embedded in a matrix which is able to adhere to the reinforcing fibres and to form between these a binding structure.

The expression “fibre” shall here be understood to encompass any organic or inorganic fibre which can be included in a textile. In the description of the present invention, the expression “reinforcing textile” is a textile including fibres operative for reinforcement of a composite material, whereas the expression “polymer matrix textile” is a textile including thermoplastic polymer fibres operative to form a matrix for embedding and stabilizing the reinforcing fibres in a composite material. The textiles are composed from threads, yarns, strings or bands which are formed from a number of individual fibres accumulated substantially in a parallel bundle, the fibres being together compressed, woven, knitted or interlaced to provide a textile which is dry, flexible and soft in normal room temperature. The fibres in such thread, yarn, string or band are substantially uniformly aligned and preferably of continuous length, which in this connection means that each individual fibre is at least as long as the length of the finished prosthetic product, or at least as long as the length of a textile or a part of textile included in the finished prosthetic product.

Inorganic fibres suitable to be included in the reinforcing textile and the present invention are glass fibres, carbon fibres, or a metal fibre, whereas suitable organic fibres can be aramid fibres (aromatic polyamide, such as Kevlar®).

Fibres which are suitable to be included in the polymer matrix textile are formed from a thermoplastic polymer material. The thermoplastic material is characterized by its ability when being heated to change from a solid state into a plastic state, from where the material again changes into a solid state upon cooling. Different from a thermosetting polymer material, a thermoplastic material can be re-shaped through repeated heating, a property which is used in the invention for individual customizing of a prosthetic product after its manufacture. Examples of suitable thermoplastic fibres in the polymer matrix textile of the present invention are: including polyethylene (PE), polyethylene terephthalate (PET), low-density polyethylene terephthalate (LPET), polybutylene therephtalate (PBT), polycarbonate (PC), acrylonitrile butadiene styrene (ABS), polyamide (PA, such as Nylon®), polypropylene (PP), polyetheretherketone (PEEK), polyetherimide (PEI), polyethersulfone (PES, such as Dacron®), polyphenylene sulfide (PPS), polyoxymethylene or polyacetal (POM), polyvinyl chloride or polyvinylidene chloride (PVC, PVDC, PVDE). Other thermoplastic polymers or co-polymers from which flexible fibres can be produced, by extrusion or other suitable process, which are not mentioned are however not excluded. Suitable fibres are also fibres that are produced from polymers which are modified in some way.

The reinforcing textile in a composite wall of a prosthetic product according to the invention may alternatively include thermoplastic fibres requiring a higher temperature to change between solid and plastic phases, then a corresponding temperature for fibres of the thermoplastic polymer matrix. One example of this embodiment is an reinforcing textile comprising polypropylene fibres (PP) having a melting point temperature of about 165° C., which when heat is applied becomes embedded in a polymer matrix textile comprising low density polyethylene terephtalate fibres (L-PET) having a melting point temperature of about 105-115° C.

Other thermoplastic fibres suitable to be included in an reinforcing textile are fibres produced from, e.g., polyethylene (PE), polyethylene terephthalate (PET), polybutylene therephtalate (PBT), polycarbonate (PC), acrylonitrile butadiene styrene (ABS), polyamide (PA, such as Nylon®), polypropylene (PP), polyetheretherketone (PEEK), polyetherimide (PEI), polyethersulfone (PES, such as Dacron®), polyphenylene sulfide (PPS), polyoxymethylene or polyacetal (POM). Other thermoplastic polymers or co-polymers from which flexible fibres can be produced, by extrusion or other suitable process, which are not mentioned are however not excluded and may be used as reinforcement fibres in a polymer matrix including thermoplastic fibres having lower melting point temperatures.

The reinforcing textile and the polymer matrix textile can be integrated into a singular textile, comprising two or more thermoplastic fibres having different melting point temperatures, or alternatively comprising one or several types of thermoplastic fibres combined with one or several types of inorganic fibres, such as glass- or carbon fibres. In a hybrid textile which is suitable for manufacture of a prosthetic product according to the invention, reinforcing fibres and matrix fibres are woven, knitted or interlaced together. In the integrated textile each individual thread, string or yarn preferably includes reinforcing fibres and matrix fibres mixed to suitable proportions and direction of fibres.

An advantageous embodiment of the invention provides that the armour and polymer matrix textiles are woven, knitted or interlaced into the circular shape of a tube or a hose. Such circular-knitted, circular-woven or circular-interlaced hose will provide advantageously a marked extensibility or elasticity, at least in a direction transversally or at an angle relative to the length direction of the tube/hose. The elasticity/extensibility will facilitate the application of the textile onto the positive copy of the body portion, and will result in a textile that is tightly applied to the surface of the mould and which follows the irregularities of the mould and its deviations from a basic geometric shape. The hoses may be endless and stored on reels, or adapted in length, and may for example have a length that constitute a multiple of the length of the manufactured prosthetic product.

The produced composite material wall advantageously includes between 30 and 80% by volume of reinforcing fibres, and between 70 and 20% by volume of thermoplastic polymer matrix fibres. The composite material wall preferably includes between 40 and 80% by volume of reinforcing fibres and between 60 and 20% by volume of thermoplastic polymer matrix fibres. From the foregoing it will be understood that when thermoplastic fibres are used also as reinforcing fibres, the textile may suitably include between 30 and 80% of such thermoplastic reinforcing fibres.

The textile/textiles may have the basic shape of a hose having a uniform diameter through its length, which preferably is the case when the textile is interlaced. Circular-knitted and circular-woven textiles may alternatively have a conical basic shape, or be knitted into other shape. Accordingly, a circular-knitted textile may have, in a local area, a number of meshes that differ from the number of meshes in other areas of the textile, such as a growing number of meshes in one direction of the hose in order to form a conical textile, e.g. Further, the textile may alternatively have, in a local area, a mesh size that differs from the mesh size in other areas of the textile. The textile may also have, in a local area, a higher fibre density and weight per area than other areas of the textile, achieved for example through individual fibres having larger diameter than other fibres in the textile, or through individual strings of fibres comprising larger numbers of individual fibres than other fibre strings in the textile. One or several of these measures can be applied in a circular-knitted textile providing higher fibre density in an axially limited region running about the hose, or in a circumferentially limited an axial region of he hose. By suitable modification of the textile in one or several ways as discussed above, a prosthetic product may be formed to have locally enforced regions, such as by increased fibre density or increased wall thickness, in certain parts of the product. A modified textile can also be advantageous and used for forming a composite material wall of uniform fibre density/wall thickness in a prosthetic product comprising major irregularities in its shape, and which cause locally a heavier extension or stretching of the textile upon application to the mould. Especially, a circular-knitted textile can be provided a high degree of shaping by using different knitting techniques, such as knitted into a curved shape or into the shape of a stocking having one or two open ends.

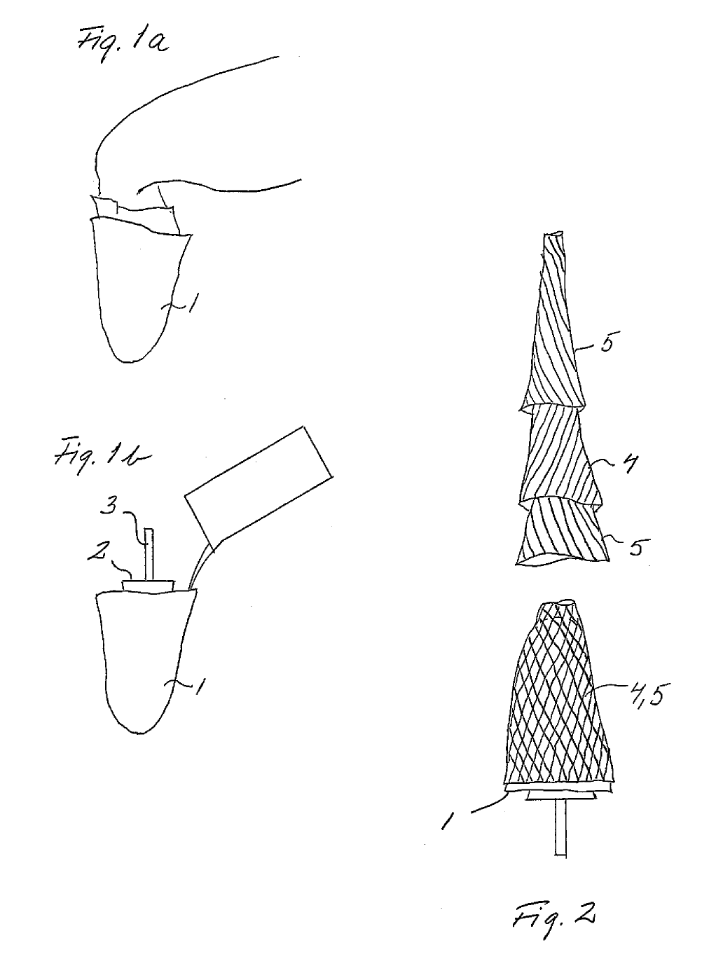

The manufacturing process will now be illustrated with reference to the drawing figures. Accordingly, FIGS. 1a and 1b illustrate schematically the production of a positive copy of a part of a body, and more precisely a copy of a stump for the manufacture of a sleeve for a lower leg prosthesis. In a first step a casting is made on the amputation stump, usually using plaster or silicon. On the resulting negative casting a positive body part copy is formed, providing the mould 1, onto which the materials included in the composite material wall are modelled. The positive mould, usually made of plaster or silicon as well, may be formed to have an inner core 2 made of other material, and a holder such as a bar 3, which is anchored in the mould core. Any required correction of the mould is performed by adding or removing material from the surface of the mould.

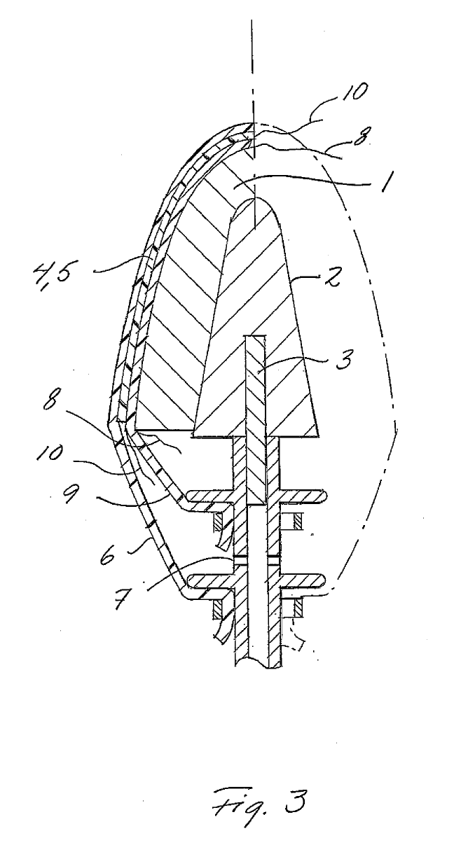

FIG. 2 illustrates the application of a hose-shaped reinforcing textile 4 and a hose-shaped thermoplastic polymer matrix textile 5 onto the mould 1, the textiles 4, 5 are preferably alternately applied one over the other to build the desired wall thickness in the composite material wall of the prosthetic product. At least one reinforcing textile and at least one thermoplastic polymer matrix textile is applied, the latter preferably being applied externally about the first mentioned textile. An advantageous embodiment includes that the reinforcing textile is applied between two thermoplastic polymer matrix textiles. Alternatively, the textiles 4, 5 may be integrated into a hose-shaped hybrid textile which is applied onto the body part copy in one or several layers.

FIG. 3 illustrates schematically in a sectional view the consolidation of the composite material wall of the prosthetic product. The mould 1, onto which the reinforcing and thermoplastic polymer matrix textiles are applied, is placed in a heating oven or corresponding heater, which generates the temperature required for melting of the fibres of the thermoplastic polymer matrix. A suitable capacity for the heater may amount to about 350° C., and at least reaching about 115°. Alternatively, heat can be supplied via the mould by means of a heat source, such as an electric resistance heater, which is located internally in a plaster mould, a silicon mould or a mould of other suitable material. A bag-shaped sleeve 6 is applied externally about the covered mould and sealed at its open end against an output in flow connection with a sub-pressure source. The sub-pressure source, such as a pump or other vacuum producing means (not shown), is operated for evacuation of air from the applied layers of reinforcing and thermoplastic polymer matrix textiles, which is caused thereby to adhere tightly towards the mould 1. Advantageously, the sub-pressure source has a capacity for establishing a negative pressure corresponding to at least 65% of complete vacuum. Heat is then applied, whereby the thermoplastic polymer matrix textile melts to be forced by the negative pressure to invade between the fibres of the reinforcing textile for embedding. The prosthetic product is then left to cool in room temperature, or is actively cooled, whereby the polymer matrix solidifies to form a homogenous, fibre reinforced composite material wall.

In order to facilitate the release of the composite material wall from the mould, a release layer 8 may be applied externally on the mould 1. The release layer 8 may consist of a bag made of synthetic material or nature fibres having a melting pint temperature above the melting point of the polymer matrix textile. A spacer sleeve 9 may additionally be applied inside the textiles of reinforcing fibres and thermoplastic fibres, in order to provide a space for applying a soft sleeve (liner) onto the amputation stump, inside the composite wall of the prosthetic product. Like the external sleeve 6, the spacer sleeve 9 may be formed of silicon, e.g. A sleeve 10 made of air permeable material or air permeable textile may additionally be arranged inside the textiles of reinforcing fibres and polymer matrix fibres, respectively, and/or inside said spacer sleeve 9, with the purpose of facilitating the evacuation of air from the textiles during consolidation.

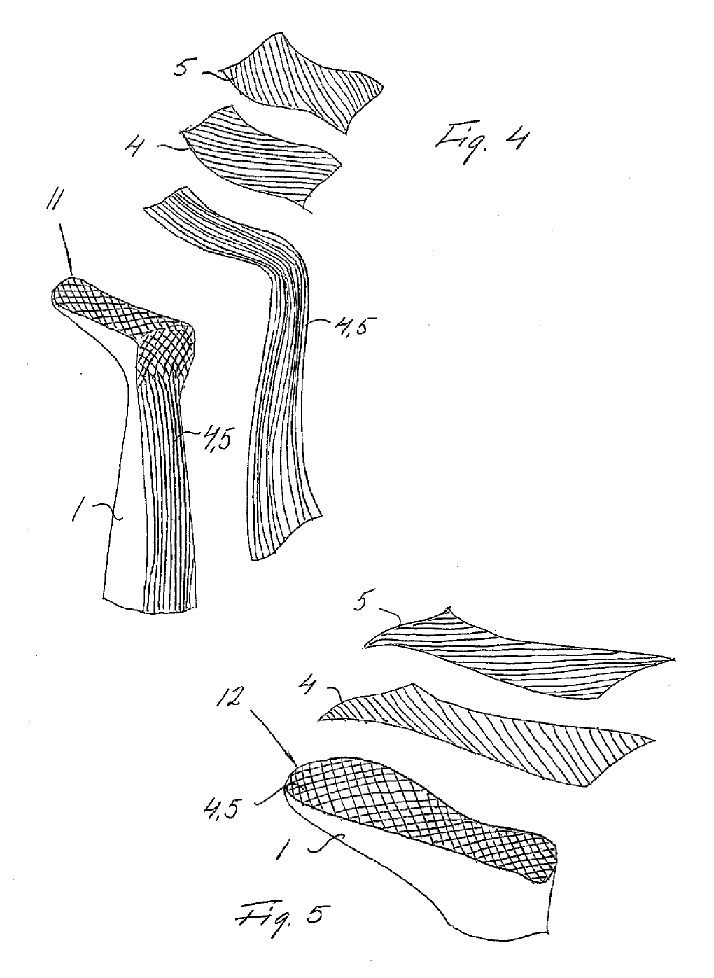

With reference to FIG. 4 an alternative embodiment is schematically illustrated in connection with the manufacture of an orthosis. Reinforcing textile 4 and thermoplastic polymer matrix textile 5 are applied onto a positive copy of a part of a body, in this case a lower leg and a foot. Like the previous example, the textiles 4, 5 can be mixed into a hybride textile including both the reinforcing fibres and the thermoplastic fibres. Alternatively, both the reinforcing fibres and the polymer matrix fibres are thermoplastic material fibres, though having different melting point temperatures. Also in this case the textiles may be shaped as hoses, or as illustrated, consist of pieces of textile which are trimmed to have suitable shape and size. Solidification of the composite material wall of the orthosis is accomplished as described above for a prosthetic sleeve.

The insert 12 illustrated in FIG. 5 can be produced in the similar way.

In all embodiments, trimming and cleaning the edges of the prosthetic product can be performed after solidification. Without specifically being shown in the drawings it will be understood that mechanical coupling means, attachments, channelling and other details can be formed by moulding into the wall of the prosthetic product, or be afterwards attached to the solidified composite material wall.

The manufacturing process results in a prosthetic product having a rigid homogenous composite material wall, which provides high strength and low weight, and which can successively be adjusted and adapted to the individual user by heating. This ability of great importance to the user separates the prosthetic product of the present invention from the known prosthetic products produced in thermosetting plastics. The prosthetic product according to the invention further provides the advantage of maintained strength in spite of low weight and reduced wall thickness, as well as reduced waste of material as compared with previous thermoplastic products. The invention thus combines the modelling ability, low weight and strength of the fibre reinforced thermosetting plastics, with the re-shaping ability of the thermoplastic. As for the manufacturing, the technology advised by the present invention results in a significant improvement of the working environment. The dry and soft textiles can be handled without working under pressure from the clock, which is reality in the shaping of preheated and softened thermoplastic materials, and avoids also the risk of burn injuries, as well as the presence of heavy smells from liquids that irritate the skin and which are associated with liquid thermosetting plastics and prepreg materials.

The invention provides a significant advantage over the prior art in that the direction of fibres is fully visible in the dry and soft textiles when applying the textile/textiles onto the mould. In result, complete control over the direction of reinforcing fibres in the finished composite material wall is provided. The invention thus makes possible a dedicated distribution of reinforcing fibres in the wall of the prosthetic product, with respect to fibre density and fibre direction. This way prosthetic products may be formed, which are adapted specifically to resist different types of loads, and even to provide enhanced resistance against a specific type of load in a certain region of the prosthetic product, whereas other regions of the product are adapted to resist other types of loads, or to provide locally an increased flexibility or rigidity. The loads that typically are applied in prosthetic products are axial and radial pressures and/or tension loads, bending, or shear.

Especially, the enhanced ability to control the direction of reinforcing fibres permits the manufacture of prosthetic products which have a favourable relation of strength/weight. Especially, the reinforcing textile may be controlled when applied to have a first fibre direction which extends mainly in the line of forces of the load that is applied to the prosthetic product during use thereof, and a second fibre direction which mainly extends transversely to the line of forces of the load that is applied to the prosthetic product during use thereof. Alternatively, or in combination with the aforesaid, the reinforcing textile can have first and second fibre directions which extend at an angle to the line of forces of the load that is applied to the prosthetic product during use thereof.

As described above, a prosthetic product is formed to have a homogenous, fibre reinforced composite material wall which is afterwards adaptable to the individual user. The prosthetic product can be shaped into a sleeve element or a frame element which is shaped for partial or complete enclosure of an amputation stump, and which operates as a means for anchoring the prosthesis to the amputation stump.

The prosthetic product may also be comprised in an orthosis or constitute the orthosis, such as in the form of a frame element with a partially broken away wall that encloses one or several parts of the body and which operates as a means for anchoring the orthosis to the body. The anchoring means of the orthosis transfers loads between the body part/body parts and the orthosis. The orthosis corrects a deformed body part, or limits the motions of one or several body parts, or stabilizes or provides relief to one or several body parts, such as neck, back, knee- or ankle joint, arm, wrist, etc.

The prosthetic product may also constitute an insert for a shoe, which insert partly encloses a foot from underneath and is operative for preventing, reducing or removing strain injuries, and improves the balance and coordination, which leads to less fatigue in feet and legs. Theses inserts may also be shaped to correct deformations and incorrect positions, as well as for providing relief of a foot in case of wounds or pain.

The skilled person will realize that modifications are possible within the scope of the claimed solution, and that details of the embodiments may be separately applied to the features of superior claims also in combinations other than those supplied, while still maintaining the benefits or the partial benefit of the solution provided by the invention.

1. A prosthetic product having a composite material wall moulded into the shape of a part of a body, comprising reinforcement fibres embedded in a thermoplastic polymer matrix, characterized in that the wall of the prosthetic product is composed of, on one hand, a reinforcing textile including continuous length fibres, and on the other hand, a textile matrix including thermoplastic polymer fibres, preferably of continuous length, which are solidified together into a composite on a positive copy of the body part and under the supply of heat and pressure.

2. The prosthetic product of claim 1, characterized in that the composite material wall of the prosthetic product is composed of between 30 and 80 percents by volume of reinforcing fibres, and between 70 and 20 percents by volume of thermoplastic polymer matrix fibres, preferably between 40 and 80 percents by volume of reinforcing fibres and between 60 and 20 percents by volume of thermoplastic polymer matrix fibres.

3. The prosthetic product according to claim 1 or 2, characterized in that the reinforcing textile and/or the thermoplastic polymer matrix textile is interlaced, woven, or knitted.

4. The prosthetic product according to claim 1, characterized in that reinforcing fibres and thermoplastic polymer matrix fibres are together woven, or together knitted, or together interlaced into a mixed textile.

5. (canceled)

6. The prosthetic product according to claim 1, characterized in that the fibres of the reinforcing textile mainly comprises at least one fibre selected from the group of fibres consisting of glass fibres, carbon fibres, and aramid fibres.

7. The prosthetic product according to claim 1, characterized in that the reinforcing textile and the polymer matrix textile both includes thermoplastic polymer fibres.

8. The prosthetic product according to claim 7, characterized in that the fibre of the reinforcing textile comprises at least one fibre selected from the group of fibres consisting of polyethylene (PE), polyethylene terephthalate (PET), polybutylene therephtalate (PBT), polycarbonate (PC), acrylonitrile butadiene styrene (ABS), polyamide (PA, such as Nylon®), polypropylene (PP), polyetheretherketone (PEEK), polyetherimide (PEI), polyethersulfone (PES, such as Dacron®), polyphenylene sulfide (PPS), polyoxymethylene, and polyacetal (POM).

9. (canceled)

10. The prosthetic product according to claim 7, characterized in that the fibre of the polymer matrix textile comprises at least one fibre selected from the group of fibres consisting of polyethylene (PE), polyethylene terephthalate (PET), low-density polyethylene terephthalate (LPET), polybutylene therephtalate (PBT), polycarbonate (PC), acrylonitrile butadiene styrene (ABS), polyamide (PA, such as Nylon®), polypropylene (PP), polyetheretherketone (PEEK), polyetherimide (PEI), polyethersulfone (PES, such as Dacron®), polyphenylene sulfide (PPS), polyoxymethylene or polyacetal (POM), polyvinyl chloride, and polyvinylidene chloride (PVC, PVDC, PVDE).

11. The prosthetic product according to claim 1, characterized in that the composite material wall is composed of at least one reinforcing textile which is embedded between thermoplastic polymer matrix textiles.

12. The prosthetic product according to claim 1, characterized in that the composite material wall is composed of several layers wherein every other layer includes a reinforcing textile and the layer between includes a polymer matrix textile.

13. The prosthetic product according to claim 1, characterized in that at least one of the reinforcing textile and the polymer matrix textile includes fibres that are interlaced, or woven, or knitted to the form of a hose.

14. The prosthetic product of claim 1, characterized in that prosthetic product is selected from a group comprising a sleeve having coupling means thereon for connection to an artificial leg, a sleeve having coupling means thereon for connection to an artificial arm, an orthosis, and an insert.

15.-19. (canceled)

20. The prosthetic product according to claim 1, characterized in that the prosthetic product is shaped to resist loads in form of pressure, and/or tension, and/or bending, and/or shear, and in that the reinforcing textile has a first fibre direction extending mainly in the line of force of the main load which is applied to the prosthetic product in use, and a second fibre direction mainly extending transversally to the line of force of the main load which is applied to the prosthetic product in use.

21. (canceled)

22. The prosthetic product according to claim 1, characterized in that the prosthetic product is shaped to resist loads in form of pressure, and/or tension, and/or bending, and/or shear, and in that the reinforcing textile has first and second fibre directions extending angularly relative to the line of force of the main load which is applied to the prosthetic product in use.

23.-31. (canceled)

32. A process for the manufacture of a prosthetic product comprising a composite material wall moulded into the shape of a part of a body, comprising the following manufacturing steps:
providing a mould onto which the composite material wall is to be formed;
applying a reinforcing textile including continuous length fibres onto the mould;
applying a matrix textile including thermoplastic polymer fibres preferably of continuous length onto the mould, at least externally of the reinforcing textile, and
melting the polymer matrix fibres for embedding of the reinforcing textile fibres in a thermoplastic polymer matrix, followed by cooling and consolidation of the composite material wall.

33. The process of claim 32, characterized in that the composite material wall of the prosthetic product is formed by alternating layers of reinforcing textile and thermoplastic polymer matrix textile onto the mould in the construction of a multi layered prosthetic product wall, which through melting and cooling of the Polymer matrix textile is consolidated into a homogenous composite material wall having the shape of the mould.

34. (canceled)

35. The process of claim 32 or 33, characterized in that the thermoplastic polymer matrix textile is melted while pressure is applied there about to act inwardly, towards the mould wherein said pressure is a negative pressure generated inside an elastic sleeve which is arranged to enclose the mould and the reinforcing and polymer matrix textiles, respectively, applied onto the mould.

36.-37. (canceled)

38. The process of claim 32, further comprising applying an elastic sleeve of air permeable material or an air permeable textile onto the mould, internally of the textiles of reinforcing fibres and thermoplastic polymer fibres, respectively.

39. The process of claim 38, further comprising applying an elastic spacer sleeve onto the mould, internally of the textiles of reinforcing fibres and thermoplastic polymer fibres, respectively, and externally or internally of the air permeable sleeve.

40.-51. (canceled)
